(19) United States
(12) Patent Application Publication
(10) Pub. No.: US 2021/0393545 A1
(43) Pub. Date: Dec. 23, 2021

(54) NATURAL PRODUCT COMPOSITION FOR STIMULATING HAIR GROWTH

(71) Applicant: Robert John PETCAVICH, The Woodlands, TX (US)

(72) Inventor: Robert John PETCAVICH, The Woodlands, TX (US)

(21) Appl. No.: 17/281,212

(22) PCT Filed: Sep. 30, 2019
(86) PCT No.: PCT/US2019/053875

§ 371 Date: Mar. 29, 2021

Related U.S. Application Data
(60) Provisional application No. 62/739,168, filed on Sep. 29, 2018.
(60) Provisional application No. 62/907,715, filed on Sep. 29, 2019.

(51) Int. Cl. A61K 31/045; A61K 31/506; A61K 36/53; A61K 35/57; A61P 17/14

(52) U.S. Cl. CPC A61K 31/045; A61K 31/506; A61P 17/14; A61K 35/57; A61K 36/53

(57) ABSTRACT

The present invention discloses a novel combination of a natural product and minoxidil for the treatment of hair loss and regrowth. In the preferred embodiment, Sandalore® and its analogs are combined with minoxidil and formulated into a solution for the treatment and regrowth of mammalian hair loss. In another embodiment, Sandalore® and its analogs are combined with rosemary oil, emu oil or a combination thereof.

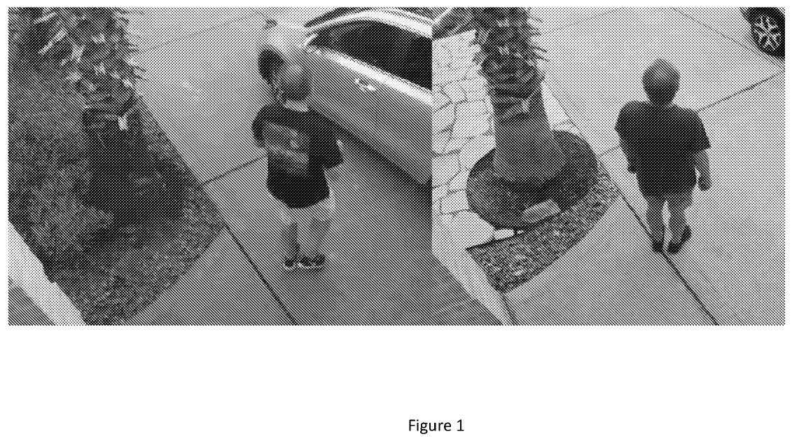

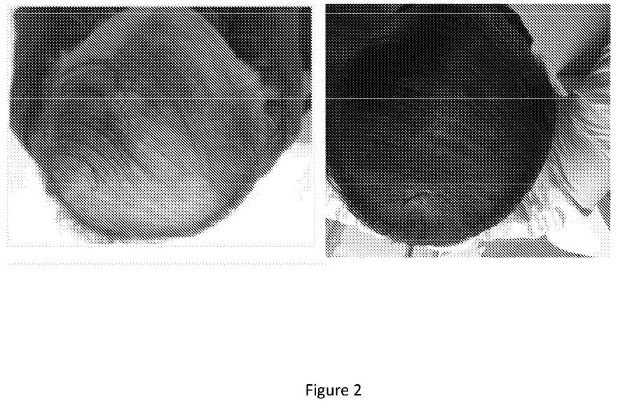

This application claims priority to and the benefit of U.S. Provisional Patent Application No. 62/907,715 filed Sep. 29, 2019 and U.S. Provisional Patent Application No. 62/739,168 filed Sep. 29, 2018, the entire contents of both are incorporated by reference herein.

### BACKGROUND OF THE INVENTION

Dermatologists recognize many different types of hair loss, the most common by far being “alopecia” wherein human males begin losing scalp hair at the temples and on the crown of the head. While this type of hair loss is mostly confined to males, hence its common name “male pattern baldness,” it is not unknown in women. No known cure has yet been found despite continuing attempts to discover one. For the purposes of the present invention, it is necessary to consider various types of hair, including, terminal hairs and vellus hairs and modified terminal hairs, such as seen in eye lashes and eyebrows. Terminal hairs are coarse, pigmented, long hairs in which the bulb of the hair follicle is seated deep in the dermis. Vellus hairs, on the other hand, are fine, thin, non-pigmented short hairs in which the hair bulb is located superficially in the dermis. As alopecia progresses, a transition takes place in the area of approaching baldness wherein the hairs themselves are changing from the terminal to the vellus type.

Another factor that contributes to the result is a change in the cycle of hair growth. All hair, both human and animal, passes through a life cycle that includes three phases, namely, the anagen phase, the catagen phase and the telogen phase. The anagen phase is the period of active hair growth and, insofar as scalp hair is concerned, this lasts from 3-5 years. The catagen phase is a short transitional phase between the anagen and telogen phases which, in the case of scalp hair, lasts only 1-2 weeks. The final phase is the telogen phase which can be denominated a “resting phase” where all growth ceases and the hair eventually is shed preparatory to the follicle commencing to grow a new one. Scalp hair in the telogen phase is also relatively short-lived, some 3-4 months elapsing before the hair is shed and a new one begins to grow.

Alopecia is associated with the severe diminution of hair follicles. A bald human subject will average only about three hundred and six (306) follicles per square centimeter, whereas, a non-bald human in the same age group will have an average of four hundred and sixty (460) follicles per square centimeter. This amounts to a one-third reduction in hair follicles which, when added to the increased proportion of vellus hair follicles and the increased number of hair follicles in the telogen phase, is both significant and noticeable. Fifty percent (50%) of the hairs must be shed to produce visible thinning of scalp hair. It is thus a combination of these factors: transition of hairs from terminal to vellus, increased number of telogen hairs—some of which have been shed, and diminution and loss of hair follicles that produces “baldness.”

While a good deal is known about the results of male pattern baldness, very little is known about its cause. The cause is generally believed to be genetic and hormonal in origin although, the known prior art attempts to control it through hormone adjustment have been, for the most part, unsuccessful.

One known treatment for male pattern alopecia is hair transplantation. Plugs of skin containing hair are transplanted from areas of the scalp where hair is growing to bald areas with reasonable success; however, the procedure is a costly one in addition to being time-consuming and quite painful. Also, psycho-sociological exist and hair transplantation may be viewed as little different from wearing a wig. Furthermore, the solution is inadequate from the standpoint that it becomes a practical, if not an economic, impossibility to replace but a tiny fraction of the hair present in a normal healthy head of hair.

Other non-drug related approaches to the problem include such things as ultra-violet radiation, massage, psychiatric treatment and exercise therapy. None of these, however, has been accepted as being effective. Even such things as revascularization surgery and acupuncture have shown little, if any, promise.

By far, the most common approach to the problem of discovering a remedy for hair loss and male pattern alopecia has been one of drug therapy. Many types of drugs ranging from vitamins to hormones have been tried and only recently has there been any indication whatsoever of even moderate success. For instance, it was felt for a long time that since an androgenic hormone was necessary for the development of male pattern baldness, that either systemic or topical application of an antiandrogenic hormone would provide the necessary inhibiting action to keep the baldness from occurring. The theory was promising, but the results were uniformly disappointing.

The androgenic hormone testosterone was known, for example, to stimulate hair growth when applied topically to the deltoid area as well as when injected into the beard and pubic regions. Even oral administration was found to result in an increased hair growth in the beard and pubic areas as well as upon the trunk and extremities. While topical application to the arm causes increased hair growth, it is ineffective on the scalp and some thinning may even result. Heavy doses of testosterone have even been known to cause male pattern alopecia.

Certain therapeutic agents have been known to induce hair growth in extensive areas of the trunk, limbs and even occasionally on the face. Such hair is of intermediate status in that it is coarser than vellus, but not as coarse as terminal hair. The hair is quite short with a length of 3 cm. being about maximum. Once the patient ceases taking the drug, the hair reverts to whatever is normal for the particular site after six months to a year has elapsed. An example of such a drug is diphenylhydantoin which is an anticonvulsant drug widely used to control epileptic seizures. Hypotrichosis is frequently observed in epileptic children some two or three months after starting the drug and first becomes noticeable on the extensor aspects of the limbs and later the trunk and face. The same pattern of hypotrichosis is sometimes caused by injury to the head. As for the hair, it is often shed when the drug is discontinued but may, in some circumstances, remain.

Streptomycin is another drug that has been found to produce hypotrichosis, in much the same way as diphenylhydantoin, when administered to children suffering from tuberculous meningitis. About the same effects were observed and the onset and reversal of the hypotrichosis in relation to the period of treatment with the antibiotic leave little question but that it was the causative agent.

Two treatments have been demonstrated as showing some promise in reversing male pattern alopecia. These treatments include the use of a microemulsion cream containing both estradiol and oxandrolone as its active ingredients and the use of organic silicon. In addition to the foregoing, it has been reported in U.S. Pat. Nos. 4,139,619 and 4,968,812, that the compound minoxidil (sold under the tradename Rogaine by Johnson & Johnson, New Brunswick, N.J.) is useful for the treatment of male pattern baldness. That compound, among others, has proven to have considerable therapeutic value in the treatment of severe hypertension. It is an anti-hypertensive “vasodilator” which, as the name implies, functions to dilate the peripheral vascular system. First introduced as an oral drug to treat high blood pressure, topical solutions and foam products were introduced to prevent or treat hair loss. Dermatologists and others have recognized that prolonged vasodilation of certain areas of the human body other than the scalp sometimes result in increased hair growth even in the absence of any vasodilating therapeutic agent. For instance, increased hair growth around surgical scars is not uncommon. Similarly, arteriovenous fistula have been known to result in increased vascularity accompanied by enhanced hair growth. Externally induced vasodilation of the skin, such as, for example, by repeated biting of the limbs by the mentally retarded and localized stimulation of the shoulders by water carries has been known to bring on hypotrichosis in the affected areas. Similar techniques, such as continued periodic massage of the scalp, have been found to be totally ineffective as a means for restoring lost hair growth to the scalp. Scar tissue on the scalp inhibits rather than promotes hair growth.

Recently, it has been reported that an aromatic chemical with odor in some ways similar to sandalwood and consequently used in perfumes, emollients, and skin cleaning agents sold under the tradename Sandalore® (Givaudan Corporation S.A., Vernier, CH) has a unique mechanism of stimulating hair growth in hair follicles. It is the novel combination of Sandalore® and minoxidil in combination is effective in treating conditions such as hypotrichosis and alopecia and for growing hair in humans.

### SUMMARY OF THE INVENTION

It is, therefore, a principal object of the present invention to provide a novel and effective treatment for the stimulation of hair growth and the treatment of male pattern baldness by the combination of a natural product namely Sandalore® and minoxidil an over the counter vasodilator.

It is, therefore, a secondary object of the present invention to provide a novel cosmetic formulation for the stimulation of hair growth in a human suffering from hair loss by providing a topical formulation comprising Sandalore® and minoxidil.

It is, therefore, a principal object of the present invention to provide a novel and effective treatment for the stimulation of hair growth and the treatment of male pattern baldness by the combination of a natural products namely Sandalore®, rosemary oil extract, emu oil or a combination thereof.

Still another objective is the provision of a treatment for the stimulation of hair growth such as eyebrows, scalp and facial hair which, while effective for its intended purpose, is non-toxic and free of unwanted side effects.

An additional object of the invention herein disclosed and claimed is to provide a method for treating hair loss in men or women, including hair loss due to chemotherapy, which can be applied by the patient under medical supervision no more stringent than that demanded for other topically-administered therapeutic agents.

Finally, it is an object of this invention to enhance the growth of eyebrows, scalp hair and facial hair in a human.

### BRIEF DESCRIPTION OF THE DRAWINGS

FIG. A comprises two side-by-side photographs of Subject 1 showing the subject prior to the application of the claimed method (left) and the subject after the application of the claimed method (right); and

FIG. B comprises two side-by-side photographs of Subject 2 showing the subject prior to the application of the claimed method (left) and the subject after the application of the claimed method (right).

### DETAILED DESCRIPTION OF THE INVENTION

Alopecia (baldness) a deficiency of either normal or abnormal hair is primarily a cosmetic problem in humans. It is a deficiency of terminal hair, the broad diameter, colored hair that is readily seen, however, in a “bald” person, even though there is a noticeable absence of terminal hair, the skin does contain vellus hair which is a fine colorless hair which may require microscopic examination to determine its presence. As stated previously, this vellus hair is a precursor to terminal hair.

Drug synergism occurs when drugs can interact in ways that enhance or magnify one or more effects, or side effects, of those drugs. Negative effects of synergy are a form of contraindication such as when more than one depressant drug is used that affects the central nervous system (CNS), an example being alcohol and Valium® (Roche Products, Inc., Hamilton, BM). The combination can cause a greater reaction than simply the sum of the individual effects of each drug if they were used separately. With respect to alcohol and Valium®, the most serious consequence of drug synergy is exaggerated respiratory depression, which can be fatal if left untreated. Synergism has also been noted in describing how complex systems operate. For example, biological systems may react in a non-linear way to perturbations, so that the outcome may be greater than the sum of the individual component alterations.

In describing the present invention, synergism means that the combination of the two active drugs utilized in the methods and compositions of the invention achieves a result, e.g., stimulating the growth of hair such as eyelashes in a mammal, e.g., a human, that is greater than the result achieved when the active drugs are utilized, alone, under the same conditions. Thus, to determine the combinations that are within the scope of the present invention, one may simply compare the result achieved by the combination of the two drugs with the result achieved with each of the individual drugs alone.

In accordance with the invention as described herein, there is provided a method for enhancing hair growth in a mammal in need thereof which comprises administering to the mammal a synergistically effective amount of Sandalore® and minoxidil. Thus, in accordance with the present invention, synergistically effective amounts of Sandalore® and minoxidil are used to stimulate the conversion of vellus hair to growth as terminal hair as well as increase the rate of growth of terminal hair.

Olfactory receptors are expressed by different cell types throughout the body and regulate physiological cell functions beyond olfaction. In particular, the olfactory receptor OR2AT4 has been shown to stimulate keratinocyte proliferation in the skin (Cheret J et al., Nature Comm, 9(3624):1-12). It has been shown that the epithelium of human hair follicles, particularly the outer root sheath, expresses OR2AT4, and that specific stimulation of OR2AT4 by Sandalore® prolongs human hair growth ex vivo by decreasing apoptosis and increasing production of the anagen-prolonging growth factor IGF-1. In contrast, co-administration of the specific OR2AT4 antagonist Phenirat® (Symrise AG, Holzminden, DE) and silencing of OR2AT4 inhibits hair growth. Studies have found that human hair follicles can engage in olfactory receptor-dependent chemo-sensation or stimulation and require OR2AT4-mediated signaling to sustain their growth, suggesting that olfactory receptors may serve as a target in hair loss therapy. This is indeed the case as discovered by the present inventor with administration of the synergistic composition disclosed herein on various volunteers to re-grow hair on the scalp. Sandalore® (chemical name 3-methyl-5(2,2,3-Trimethylcyclepent-3-en-1-yl) pentan-2-ol) having the structure shown below is part of the novel composition disclosed in the present invention:

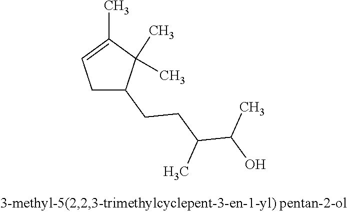

In addition to Sandalore®, analogs and derivatives of the molecule can also be utilized in the present inventions such as 3,3-dimethyl-5-(2,2,3-trimethylcyclopent-3-en-1-yl) pentan-2-ol, 4-methyl-6-(2,2,3-trimethylcyclopent-3-en-1-yl) hexan-3-ol, 5-(2,2,3-trimethylcyclopent-3-3n-1-yl) pentan-2-ol, 4-methyl-5-(2,2,3-trimethylcyclopent-3-en-1-yl) pentan-2-ol, 6-(2,2,3-trimethylcyclopent-3-en-1-yl) hexan-3-ol, 2-methyl-4-(2,2,3-trimethyl-1-cyclopent-3-enyl) butan-1-ol (sold under the tradename Brahmanol® by Symrise AG Aktiengesellschaft, Holzminden, DE) or 2-methyl-4-(2,2,3-trimethylcyclopent-3-3n-1-yl) butan-1-ol.

Another compound of the present invention composition, minoxidil (6-Piperidin-1-ylpyrinidine-2,4,diamine-3-oxide) (Johnson & Johnson, New Brunswick, N.J.) is a vasodilator medication known for its ability to slow or stop hair loss and promote hair regrowth. It is available over the counter for treatment of androgenic alopecia, among other baldness treatments, but measurable changes disappear within months after discontinuation of treatment. Minoxidil has the structure shown below and may include physiologically acceptable salts, prodrugs and isomers.

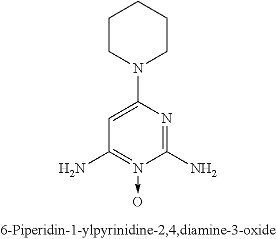 [SMILES: Nc1cc(N2CCCCC2)nc(N)[n+]1[O-]]

In another embodiment of the claimed invention, instead of minoxidil, rosemary oil extract may be substituted for minoxidil. Inhibition of testosterone 5 alpha reductase is one of the strategies postulated for the treatment of baldness. Recently it has been reported in the literature that rosemary oil extract is an antagonist for that enzyme and as a result a possible natural therapeutic target for the treatment of all forms of hair loss. Rosemary oil consist of several active compounds, but carnosic acid, in particular, was found to be the active ingredient in suppressing 5 alpha reductase activity. Therefore, it is utilized in the present invention as a co-ingredient of the preferred embodiment. The chemical structure of carnosic acid is below:

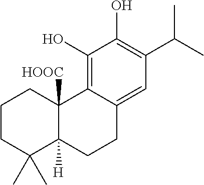 [SMILES: CC(C)c1cc2c(c(O)c1O)[C@@]1(C(=O)O)CCCC(C)(C)[C@@H]1CC2]

Another compound of the present invention composition, emu oil that has the composition shown in Table 1 and may include antioxidants.

TABLE 1

| Fatty Acid | % by weight |
|---|---|
| Lauric | 0.1 |
| Myristic | 0.3 |
| Palmitic | 21 |
| Stearic | 10 |
| Palmitoleic | 4 |
| Oleic | 46 |
| Linoleic | 16 |
| Linolenic | 2 |

Emu oil has three beneficial attributes in the present invention. The first is that it is an excellent carrier of active oil-based ingredients. The second it has the most unusual property of being able to quickly penetrate through all the layers of the skin and carry active ingredients into the bloodstream, and third the fatty acid composition is almost identical to human skin secretions which makes it very biocompatible. Emu oil by itself has been reported to stimulate hair growth by enhancing the health and longevity of hair follicles.

In accordance with one aspect of the invention, the natural compounds, i.e. Sandalore®, rosemary oil and emu oil, alone or in combination, may or may not be mixed with or without a dermatologically compatible vehicle or carrier. The vehicle which may be employed for preparing compositions of this invention may comprise, for example, aqueous solutions such as e.g., physiological salines, alcohols, glycols, oil solutions, foams, shampoos or ointments and may contain surfactants and wetting agents. The vehicle furthermore may contain dermatologically—compatible preservatives such as e.g., benzalkonium chloride, surfactants like e.g., polysorbate 80, liposomes or polymers, for example, methyl cellulose, polyvinyl alcohol, polyvinyl pyrrolinone and hyaluronic acid; these may be used for increasing the viscosity.

In accordance with one aspect of the invention, the natural compound, i.e. Sandalore® and minoxidil or rosemary oil, and emu oil (or a combination thereof), are mixed with a dermatologically compatible vehicle or carrier. The vehicle which may be employed for preparing compositions of this invention may comprise, for example, aqueous solutions such as e.g., physiological saline, alcohols, glycols, oil solutions or ointments and, in some examples, contain excipients, such as surfactants and wetting agents. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolinone and hyaluronic acid; these may be used for increasing the viscosity. It should be appreciated that the present invention may contain other components for topical drugs as known in the art, such as those taught by Chang, R-K et al., (2012), AAPS J, 15(1):41-52, the contents of which is incorporated herein.

The invention is also related to dermatological compositions for topical treatment for the stimulation of hair growth, which comprise an effective hair growth stimulating amount of Sandalore®, minoxidil or rosemary and/or emu oil (or a combination thereof) and a dermatologically compatible carrier. Effective amounts of the active compounds may be determined by one of ordinary skill in the art but will vary depending on the frequency of application and desired result, and Sandalore® or Brahmanol® will range from about 0.0000001 to about 10%, by weight, of the dermatological composition, preferably from about 0.001 to about 10%, by weight, of total dermatological composition, more preferably from about 0.01 to about 5% by weight of the composition and minoxidil will range from about 0.001 to about 10%, by weight, of the dermatological composition, preferably from about 0.01 to about 10%, by weight, of the composition. The rosemary oil from 0.00001 to 10% by weight of the dermatological composition, preferably from about 0.001 to about 10%, by weight, of total dermatological composition, more preferably from about 0.01 to about 5% by weight of the composition and emu oil will range from about 1 to about 99%, by weight, of the dermatological composition, preferably from about 10 to about 95%, by weight, of the composition.

For topical use on the skin and the scalp, the composition can be advantageously formulated using, foams, sprays, ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betaine, chlorhexidine, benzalkonium chloride, and the like. Various matrices for slow release delivery may also be used. To achieve the daily amount of medication depending on the formulation, the compound, upon the advice of a consulting medical professional, may be administered once or several times daily with or without antioxidants.

In addition to the disclosed methods of this invention, there is provided a composition of Sandalore® and minoxidil or rosemary oil and emu oil (alone or in combination thereof) in a vehicle for topical application to the skin of a mammal whereby the combination of Sandalore® and minoxidil (or rosemary oil and emu oil) produces a faster onset of hair growth in humans or animals and wherein said composition brings about a synergistic result of faster onset of hair growth as compared to compositions comprising Sandalore® and minoxidil, Sandalore® and rosemary oil and emu oil, or Sandalore®, minoxidil, rosemary oil and emu oil alone.

Typically, the novel composition is applied repeatedly for a sustained period topically on the part of the body to be treated, for example, the eyebrows, skin or scalp. The preferred dosage regimen will involve regular, such as daily, administration for a period of treatment of at least one month, more preferably at least three months, and most preferably at least six months.

The invention is further illustrated by the following non-limiting example.

### Example 1

An aqueous solution containing Sandalore® and minoxidil is prepared as follows:

Dissolve Sandalore® (Givaudan Corporation S.A., Vernier, CH) 0.05% by weight and minoxidil (Johnson & Johnson, New Brunswick, N.J.) 5% by weight in water and after both are completely dissolved, add propylene glycol (PEG) (MedLab Supply, Plantation, Fla.), alcohol and wetting agent Triton X™ (4-(1,1,3,3-Tetramethylbutyl)phenyl-polyethylene glycol)(Millipore Sigma, St. Louis, Mo.) and is sterilize the resulting solution by filtration. The solution is aseptically filled into sterile containers. The composition so prepared can be used in the topical treatment of baldness by application to the scalp daily. The amount and frequency of use can vary for the desired hair regrowth results, upon consultation with a patient's medical professional. Preferably from 3-5 milliliters is applied to the scalp once or twice per day over a duration of several months.

### Example 2

A solution containing Sandalore®, rosemary oil and emu it is prepared as follows:

Dissolve Sandalore® (Givaudan Corporation S.A., Vernier, CH) 0.05% by weight and rosemary oil 1% by weight and emu oil 5% by weight in propylene glycol (PEG) and alcohol and an optional wetting agent such at Triton X™ (4-(1,1,3,3-Tetramethylbutyl)phenyl-polyethylene glycol)(Millipore Sigma, St. Louis, Mo.) and thereafter sterilizing the resulting solution by filtration. The solution is aseptically filled into sterile containers. The composition so prepared can be used in the topical treatment of baldness by application to the scalp daily. The amount and frequency of use can vary for the desired hair regrowth results upon the advice of a medical professional. Preferably, from 1-3 milliliters is applied to the scalp once or twice per day over a duration of several months.

### Example 3

A solution containing Brahmanol®, rosemary oil and emu oil is prepared as follows:

Dissolve 2-methyl-4-(2,2,3-trimethyl-1-cyclopent-3-enyl) butan-1-ol (Brahmanol®, Symrise AG Aktiengesellschaft, Holzminden, DE) 1.0% by weight, rosemary oil 1% and emu oil 5% by weight are dissolved in propylene glycol and/or alcohol and an optional wetting agent such at Triton X™ (4-(1,1,3,3-Tetramethylbutyl)phenyl-polyethylene glycol)(Millipore Sigma, St. Louis, Mo.) and the resulting solution is sterilized by filtration. The solution is aseptically filled into sterile containers. The composition can be used in the topical treatment of baldness by application to the scalp daily. The amount and frequency of use can vary for the desired hair regrowth results. Preferably, from 1-3 milliliters is applied to the scalp once or twice per day over a duration of several months.

Referring now to FIG. A, a healthy volunteer exhibiting male pattern baldness (Subject 1) began topically applying 3-5 milliliters of the claimed pharmaceutical composition, as discussed above, once or twice per day to his scalp. The photograph on the left depicts the severity of Subject 1's hair loss on the day said subject began using the claimed composition, i.e. 14 Sep. 2018. The photograph on the right depicts the restoration of Subject 1's hair after discontinuing use on 24 Jun. 2019. FIG. B, also depicts the effects of topically applying 3-5 milliliters of the claimed pharmaceutical composition, as discussed above, once or twice per day to his scalp to a healthy volunteer exhibiting male pattern baldness (Subject 2). The photograph on the left depicts the severity of Subject 2's hair loss on the day said subject began using the claimed composition in December 2018 and the photograph on the right depicts the restoration of Subject 2's hair as of March 2019.

As referred to herein, “about”, when used to modify an amount, generally means±5%.

Although the present technology has been described in relation to a particular embodiment thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

What is claimed is:

1. A cosmetic formulation comprising an effective amount of 3-methyl-5(2,2,3-trimethylcyclepent-3-en-1-yl) pentan-2-ol or an analog thereof, an effective amount of (6-piperidin-1-ylpyrinidine-2,4, diamine-3-oxide) and a carrier.

2. The formulation according to claim 1, wherein said formulation is used to cosmetically improve the appearance of a human user thereof experiencing a loss of hair.

3. The formulation according to claim 2, wherein said hair loss occurs on the scalp of said user.

4. The formulation according to claim 3, wherein said formulation stimulates the growth of hair on the scalp of the user.

5. The formulation according to claim 1, wherein said carrier is dermatologically-compatible with human skin.

6. The formulation according to claim 5, wherein said formulation is applied topically to the scalp of the user.

7. The formulation according to claim 5, wherein said carrier is an aqueous solution.

8. The formulation according to claim 7, wherein said carrier is selected from the group consisting of physiological saline, glycol, oil solutions, gelling agents and combinations thereof.

9. The formulation according to claim 8, further comprising one or more excipient.

10. The formulation according to claim 9, wherein said excipient may be a dermatologically-compatible surfactant, dermatologically-compatible wetting agent, dermatologically-compatible preservative or a dermatologically-compatible viscosity agent.

11. The formulation according to claim 10, wherein said dermatologically-compatible preservative is selected from the group consisting of benzalkonium chloride, methyl-parahydroxybenzoic acid, propyl-parahydroxybenzoic acid, butyl-parahydroxybenzoic acid, betaine, chlorhexidine, benzalkonium chloride and combinations thereof.

12. The formulation according to claim 10, wherein said dermatologically-compatible surfactant is selected from the group consisting of polysorbate 80, one or more liposomes, one or more polymers and combinations thereof.

13. The formulation according to claim 12, wherein said dermatologically-compatible surfactant is a polymer.

14. The formulation according to claim 13, wherein said polymer is selected from the group consisting of methyl cellulose, polyvinyl alcohol, polyvinyl pyrrolinone, hyaluronic acid and combinations thereof.

15. The formulation according to claim 14, wherein said dermatologically-compatible polymer is used to increase the viscosity of said cosmetic formulation.

16. The formulation according to claim 8, wherein said cosmetic formulation is formulated as a foam, spray, ointment, cream, liniment or patch.

17. The formulation according to claim 16, wherein said cosmetic formulation is stored and dispensed from a container selected from the group consisting of a bottle with a cap, a deformable tube, an aerosol can, a pump sprayer and a jar.

18. The formulation according to claim 17, wherein said storage container is a bottle with a cap further comprising a dropper inserted into said cap.

19. The formulation according to claim 1, further comprising a dermatologically-compatible antioxidant.

20. The formulation according to claim 19, wherein said dermatologically-compatible antioxidant is butylated hydroxyanisole or butylated hydroxytoluene.

21. The formulation according to claim 1, wherein said cosmetic formulation comprises 3-methyl-5(2,2,3-trimethylcyclepent-3-en-1-yl) pentan-2-ol from about 0.0000001 to about 10%, by weight of the formulation, preferably from about 0.001 to about 10%, by weight of the formulation, more preferably from about 0.01 to about 5% by weight of the formulation.

22. The formulation according to claim 1, wherein said cosmetic formulation comprises (6-piperidin-1-ylpyrinidine-2,4, diamine-3-oxide) from about 0.001 to about 10% by weight of the formulation, preferably from about 0.01 to about 10% by weight, of the formulation.

23. The formulation according to claim 1, wherein said analog 3-methyl-5(2,2,3-trimethylcyclepent-3-en-1-yl) pentan-2-ol) is selected from the group consisting of 3,3-dimethyl-5-(2,2,3-trimethylcyclopent-3-en-1-yl)pentan-2-ol, 4-methyl-6-(2,2,3-trimethylcyclopent-3-en-1yl)hexan-3-ol, 5-(2,2,3-trimethylcyclopent-3-3n-1-yl)pentan-2-ol, 4-methyl-5-(2,2,3-trimethylcyclopent-3-en-1-yl)pentan-2-ol, 6-(2,2,3-trimethylcyclopent-3-en-1-yl)hexan-3-ol, 2-methyl-4-(2,2,3-trimethyl-1-cyclopent-3-enyl)butan-1-ol, 2-methyl-4-(2,2,3-trimethylcyclopent-3-3n-1-yl)butan-1-ol and combinations thereof.

24. The formulation according to claim 1, wherein said user has undergone or is undergoing hair loss treatments selected from the group consisting of hair transplantation, ultra-violet radiation, massage, psychiatric treatment and exercise therapy or has been administered various drugs selected from the group consisting of a systemic or topical antiandrogenic hormone, testosterone, diphenylhydantoin, streptomycin, estradiol and oxandrolone.

25. The formulation according to claim 1, wherein said formulation is applied to the scalp, eyebrows or face.

26. The formulation according to claim 25, wherein said user is suffering hair loss due to chemotherapy treatments.

27. The formulation according to claim 1, wherein said formulation is non-toxic and does not cause unwanted side effects.

28. A pharmaceutical formulation comprising an effective amount of 3-methyl-5(2,2,3-trimethylcyclepent-3-en-1-yl) pentan-2-ol or an analog thereof, an effective amount of (6-piperidin-1-ylpyrinidine-2,4, diamine 3-oxide) and a carrier.

29. The formulation according to claim 28, wherein said formulation is used to cosmetically improve the appearance of a human user thereof experiencing a loss of hair.

30. The formulation according to claim 29, wherein said hair loss occurs on the scalp of the user.

31. The formulation according to claim 30, wherein said formulation stimulates the growth of hair on the scalp of the user.

32. The formulation according to claim 28, wherein said carrier is dermatologically compatible with human skin.

33. The formulation according to claim 32, wherein said formulation is applied topically to the scalp of the user.

34. The formulation according to claim 32, wherein said carrier is an aqueous solution.

35. The formulation according to claim 34, wherein said carrier is selected from the group consisting of physiological saline, glycol, oil solutions, gelling agents and combinations thereof.

36. The formulation according to claim 35, further comprising one or more excipient.

37. The formulation according to claim 36, wherein said excipient may be a dermatologically-compatible surfactant, dermatologically-compatible wetting agent, dermatologically-compatible preservative or dermatologically-compatible viscosity agent.

38. The formulation according to claim 37, wherein said dermatologically-compatible preservative is selected from the group consisting of benzalkonium chloride, methyl-parahydroxybenzoic acid, propyl-parahydroxybenzoic acid, butyl-parahydroxybenzoic acid, betaine, chlorhexidine, benzalkonium chloride and combinations thereof.

39. The formulation according to claim 37, wherein said dermatologically-compatible surfactant is selected from the group consisting of polysorbate 80, one or more liposomes, one or more polymers and combinations thereof.

40. The formulation according to claim 39, wherein said dermatologically-compatible surfactant is a polymer.

41. The formulation according to claim 40, wherein said polymer is selected from the group consisting of methyl cellulose, polyvinyl alcohol, polyvinyl pyrrolinone, hyaluronic acid and combinations thereof.

42. The formulation according to claim 41, wherein said dermatologically-compatible polymer is used to increase the viscosity of said pharmaceutical formulation.

43. The formulation according to claim 35, wherein said pharmaceutical formulation is formulated as a foam, spray, ointment, cream, liniment or patch.

44. The formulation according to claim 43, wherein said pharmaceutical formulation is stored and dispensed from a container selected from the group consisting of a bottle with a cap, a deformable tube, an aerosol can, a pump sprayer and a jar.

45. The formulation according to claim 44, wherein said storage container is a jar with cap further comprising a dropper inserted into said cap.

46. The formulation according to claim 28, further comprising a dermatologically-compatible antioxidant.

47. The formulation according to claim 46, wherein said dermatologically-compatible antioxidant is butylated hydroxyanisole or butylated hydroxytoluene.

48. The formulation according to claim 28, wherein said pharmaceutical formulation comprises 3-methyl-5(2,2,3-trimethylcyclepent-3-en-1-yl) pentan-2-ol from about 0.0000001 to about 10% by weight, of the dermatological formulation, preferably from about 0.001 to about 10%, by weight of the formulation, more preferably from about 0.01 to about 5% by weight of the formulation.

49. The formulation according to claim 28, wherein said pharmaceutical formulation comprises (6-piperidin-1-ylpyrinidine-2,4, diamine-3-oxide) from about 0.001 to about 10% by weight, of the formulation, preferably from about 0.01 to about 10% by weight, of the formulation.

50. The formulation according to claim 28, wherein said analog 3-methyl-5(2,2,3-trimethylcyclepent-3-en-1-yl) pentan-2-ol is selected from the group consisting of 3,3-dimethyl-5-(2,2,3-trimethylcyclopent-3-en-1-yl)pentan-2-ol, 4-methyl-6-(2,2,3-trimethylcyclopent-3-en-1yl)hexan-3-ol, 5-(2,2,3-trimethylcyclopent-3-3n-1-yl)pentan-2-ol, 4-methyl-5-(2,2,3-trimethylcyclopent-3-en-1-yl)pentan-2-ol, 6-(2,2,3-trimethylcyclopent-3-en-1-yl)hexan-3-ol, 2-methyl-4-(2,2,3-trimethyl-1-cyclopent-3-enyl)butan-1-ol, 2-methyl-4-(2,2,3-trimethylcyclopent-3-3n-1-yl)butan-1-ol and combinations thereof.

51. The formulation according to claim 28, wherein said user has undergone or is undergoing hair loss treatments selected from the group consisting of hair transplantation, ultra-violet radiation, massage, psychiatric treatment and exercise therapy or has been administered various drugs selected from the group consisting of a systemic or topical antiandrogenic hormone, testosterone, diphenylhydantoin, streptomycin, estradiol and oxandrolone.

52. The formulation according to claim 28, wherein said formulation is applied to the scalp, eyebrows or face.

53. The formulation according to claim 52, wherein said user is suffering hair loss due to chemotherapy treatments.

54. The formulation according to claim 28, wherein said formulation is non-toxic and does not cause unwanted side effects.

55. A method to improve the cosmetic appearance of a human comprising the step of administering the formulation according to any one of claims 1 to 27.

56. The method according to claim 55, wherein said human is experiencing hair loss.

57. The method according to said 56, wherein said formulation enhances hair growth in said human.

58. The method according to said 56, wherein said formulation stimulates human olfactory receptor OR2AT4.

59. A method of treating a patient suffering from alopecia comprising the step of administering an effective amount of the formulation according to any one of claims 28 through 54.

60. The method according to said 59, wherein said formulation enhances hair growth in said human.

61. The method according to said 59, wherein said formulation stimulates human olfactory receptor OR2AT4.

62. A cosmetic formulation comprising an effective amount of 3-methyl-5(2,2,3-trimethylcyclepent-3-en-1-yl) pentan-2-ol or an analog thereof and an effective amount of rosemary oil, emu oil or combination thereof and an optional carrier.

63. The formulation according to claim 62, wherein said formulation is used to cosmetically improve the appearance of a human user thereof experiencing a loss of hair.

64. The formulation according to claim 63, wherein said hair loss occurs on the scalp of said user.

65. The formulation according to claim 64, wherein said formulation stimulates the growth of hair on the scalp of the user.

66. The formulation according to claim 62, wherein said carrier is dermatologically-compatible with human skin.

67. The formulation according to claim 66, wherein said formulation is applied topically to the scalp of the user.

68. The formulation according to claim 65, wherein said carrier is an aqueous solution.

69. The formulation according to claim 68, wherein said carrier is selected from the group consisting of physiological saline, glycol, oil solutions, gelling agents and combinations thereof.

70. The formulation according to claim 69, further comprising one or more excipients.

71. The formulation according to claim 70, wherein said excipient may be a dermatologically-compatible surfactant, dermatologically-compatible wetting agent, dermatologically-compatible preservative or a dermatologically-compatible viscosity agent.

72. The formulation according to claim 71, wherein said dermatologically-compatible preservative is selected from the group consisting of benzalkonium chloride, methyl-parahydroxybenzoic acid, propyl-parahydroxybenzoic acid, butyl-parahydroxybenzoic acid, betaine, chlorhexidine, benzalkonium chloride and combinations thereof.

73. The formulation according to claim 71, wherein said dermatologically-compatible surfactant is selected from the group consisting of polysorbate 80, one or more liposomes, one or more polymers and combinations thereof.

74. The formulation according to claim 73, wherein said dermatologically-compatible surfactant is a polymer.

75. The formulation according to claim 74, wherein said polymer is selected from the group consisting of methyl cellulose, polyvinyl alcohol, polyvinyl pyrrolinone, hyaluronic acid and combinations thereof.

76. The formulation according to claim 75, wherein said dermatologically-compatible polymer is used to increase the viscosity of said cosmetic formulation.

77. The formulation according to claim 69, wherein said cosmetic formulation is formulated as a foam, spray, ointment, cream, liniment or patch.

78. The formulation according to claim 77, wherein said cosmetic formulation is stored and dispensed from a container selected from the group consisting of a bottle with a cap, a deformable tube, an aerosol can, a pump sprayer and a jar.

79. The formulation according to claim 78, wherein said storage container is a bottle with a cap further comprising a dropper inserted into said cap.

80. The formulation according to claim 62, further comprising a dermatologically-compatible antioxidant.

81. The formulation according to claim 70, wherein said dermatologically-compatible antioxidant is butylated hydroxy anisole or butylated hydroxytoluene.

82. The formulation according to claim 62, wherein said cosmetic formulation comprises 3-methyl-5(2,2,3-trimethylcyclepent-3-en-1-yl) pentan-2-ol from about 0.0000001 to about 10%, by weight of the formulation, preferably from about 0.001 to about 10%, by weight of the formulation, more preferably from about 0.01 to about 5% by weight of the formulation.

83. The formulation according to claim 62, wherein said cosmetic formulation comprises 0.001% to 10% rosemary, emu oil, or a combination thereof, by weight.

84. The formulation according to claim 62, wherein said analog of 3-methyl-5(2,2,3-trimethylcyclepent-3-en-1-yl) pentan-2-ol) is selected from the group consisting of 3,3-dimethyl-5-(2,2,3-trimethylcyclopent-3-en-1-yl)pentan-2-ol, 4-methyl-6-(2,2,3-trimethylcyclopent-3-en-1yl)hexan-3-ol, 5-(2,2,3-trimethylcyclopent-3-3n-1-yl)pentan-2-ol, 4-methyl-5-(2,2,3-trimethylcyclopent-3-en-1-yl)pentan-2-ol, 6-(2,2,3-trimethylcyclopent-3-en-1-yl)hexan-3-ol, 2-methyl-4-(2,2,3-trimethyl-1-cyclopent-3-enyl)butan-1-ol, 2-methyl-4-(2,2,3-trimethylcyclopent-3-3n-1-yl)butan-1-ol and combinations thereof.

85. The formulation according to claim 62, wherein said user has undergone or is undergoing hair loss treatments selected from the group consisting of hair transplantation, ultra-violet radiation, massage, psychiatric treatment and exercise therapy or has been administered various drugs selected from the group consisting of a systemic or topical antiandrogenic hormone, testosterone, diphenylhydantoin, streptomycin, estradiol and oxandrolone.

86. The formulation according to claim 62, wherein said formulation is applied to the scalp, eyebrows or face.

87. The formulation according to claim 87, wherein said user is suffering hair loss due to chemotherapy treatments.

88. The formulation according to claim 62, wherein said formulation is non-toxic and does not cause unwanted side effects.

89. A pharmaceutical formulation comprising an effective amount of 3-methyl-5(2,2,3-trimethylcyclepent-3-en-1-yl) pentan-2-ol or an analog thereof, an effective amount of rosemary oil, emu oil or a combination thereof and an optional carrier.

90. The formulation according to claim 89, wherein said formulation is used to cosmetically improve the appearance of a human user thereof experiencing a loss of hair.

91. The formulation according to claim 90, wherein said hair loss occurs on the scalp of the user.

92. The formulation according to claim 91, wherein said formulation stimulates the growth of hair on the scalp of the user.

93. The formulation according to claim 89, wherein said carrier is dermatologically compatible with human skin.

94. The formulation according to claim 93, wherein said formulation is applied topically to the scalp of the user.

95. The formulation according to claim 93, wherein said carrier is an aqueous solution.

96. The formulation according to claim 95, wherein said carrier is selected from the group consisting of physiological saline, glycol, oil solutions, gelling agents and combinations thereof.

97. The formulation according to claim 96, further comprising one or more excipients.

98. The formulation according to claim 97, wherein said excipient may be a dermatologically-compatible surfactant, dermatologically-compatible wetting agent, dermatologically-compatible preservative or dermatologically-compatible viscosity agent.

99. The formulation according to claim 98, wherein said dermatologically-compatible preservative is selected from the group consisting of benzalkonium chloride, methyl-parahydroxybenzoic acid, propyl-parahydroxybenzoic acid, butyl-parahydroxybenzoic acid, betaine, chlorhexidine, benzalkonium chloride and combinations thereof.

100. The formulation according to claim 98, wherein said dermatologically-compatible surfactant is selected from the group consisting of polysorbate 80, one or more liposomes, one or more polymers and combinations thereof.

101. The formulation according to claim 99, wherein said dermatologically-compatible surfactant is a polymer.

102. The formulation according to claim 100, wherein said polymer is selected from the group consisting of methyl cellulose, polyvinyl alcohol, polyvinyl pyrrolinone, hyaluronic acid and combinations thereof.

103. The formulation according to claim 101, wherein said dermatologically-compatible polymer is used to increase the viscosity of said pharmaceutical formulation.

104. The formulation according to claim 96, wherein said pharmaceutical formulation is formulated as a foam, spray, ointment, cream, liniment or patch.

105. The formulation according to claim 104, wherein said pharmaceutical formulation is stored and dispensed from a container selected from the group consisting of a bottle with a cap, a deformable tube, an aerosol can, a pump sprayer and a jar.

106. The formulation according to claim 105, wherein said storage container is ajar with cap further comprising a dropper inserted into said cap.

107. The formulation according to claim 62, further comprising a dermatologically-compatible antioxidant.

108. The formulation according to claim 107, wherein said dermatologically-compatible antioxidant is butylated hydroxyanisole or butylated hydroxytoluene.

109. The formulation according to claim 62, wherein said pharmaceutical formulation comprises 3-methyl-5(2,2,3-trimethylcyclepent-3-en-1-yl) pentan-2-ol from about 0.0000001 to about 10% by weight, of the dermatological formulation, preferably from about 0.001 to about 10%, by weight of the formulation, more preferably from about 0.01 to about 5% by weight of the formulation.

110. The formulation according to claim 62, wherein said pharmaceutical formulation comprises 0.001% to 10% rosemary, emu oil, or a combination thereof, by weight.

111. The formulation according to claim 62, wherein said analog of 3-methyl-5(2,2,3-trimethylcyclepent-3-en-1-yl) pentan-2-ol is selected from the group consisting of 3,3-dimethyl-5-(2,2,3-trimethylcyclopent-3-en-1-yl)pentan-2-ol, 4-methyl-6-(2,2,3-trimethylcyclopent-3-en-1yl)hexan-3-ol, 5-(2,2,3-trimethylcyclopent-3-3n-1-yl)pentan-2-ol, 4-methyl-5-(2,2,3-trimethylcyclopent-3-en-1-yl)pentan-2-ol, 6-(2,2,3-trimethylcyclopent-3-en-1-yl)hexan-3-ol, 2-methyl-4-(2,2,3-trimethyl-1-cyclopent-3-enyl)butan-1-ol, 2-methyl-4-(2,2,3-trimethylcyclopent-3-3n-1-yl)butan-1-ol and combinations thereof.

112. The formulation according to claim 62, wherein said user has undergone or is undergoing hair loss treatments selected from the group consisting of hair transplantation, ultra-violet radiation, massage, psychiatric treatment and exercise therapy or has been administered various drugs selected from the group consisting of a systemic or topical antiandrogenic hormone, testosterone, diphenylhydantoin, streptomycin, estradiol and oxandrolone.

113. The formulation according to claim 62, wherein said formulation is applied to the scalp, eyebrows or face.

114. The formulation according to claim 113, wherein said user is suffering hair loss due to chemotherapy treatments.

115. The formulation according to claim 113, wherein said formulation is non-toxic and does not cause unwanted side effects.

116. A method to improve the cosmetic appearance of a human comprising the step of administering the formulation according to any one of claims 62 to 115.

117. The method according to claim 116, wherein said human is experiencing hair loss.

118. The method according to said 117, wherein said formulation enhances hair growth in said human.

119. The method according to said 118, wherein said formulation stimulates human olfactory receptor OR2AT4.

120. A method of treating a patient suffering from alopecia comprising the step of administering an effective amount of the formulation according to any one of claims 62 through 115.

121. The method according to said 120, wherein said formulation enhances hair growth in said human.

122. The method according to said 120, wherein said formulation stimulates human olfactory receptor OR2AT4.
